(19) United States
(12) Patent Application Publication
(10) Pub. No.: US 2021/0332445 A1
(43) Pub. Date: Oct. 28, 2021

(54) VIRUS DETECTION PIPELINE

(71) Applicant: OZ Optics Ltd., Ottawa (CA)

(72) Inventors: Osman Ugur Sezerman, Ottawa (CA); Orhan Ozcan, Ottawa (CA); Eray Sahin, Ottawa (CA); Mehmet Ömür Sezerman, Kanata (CA)

(21) Appl. No.: 17/238,466

(22) Filed: Apr. 23, 2021

(30) Foreign Application Priority Data
Apr. 23, 2020 (CA) 3078004

(51) Int. Cl. C12Q 1/70; G01N 21/80

(52) U.S. Cl. CPC C12Q 1/701; G01N 21/80

(57) ABSTRACT

Current laboratory procedures for detection of a virus using the specimens collected from upper respiratory systems involve RNA isolation from the specimen, cDNA synthesis via reverse transcription, amplification of the target region via LAMP reaction, and detection. The present invention performs alternative ways of LAMP reactions in which every single key component in the traditional system is reorganized to achieve operational LAMP protocols. The present invention skips the RNA isolation and purification steps which decrease the overall cost, and the test time by more than 20 minutes. This method can also be applicable for analysis of other viruses and some pathogens in several fields.

### CROSS-REFERENCE TO RELATED APPLICATIONS

This application claims the benefit of Canadian Application No. 3,078,004, filed Apr. 23, 2020, which is expressly incorporated by reference in its entirety, including any references contained therein.

### TECHNICAL FIELD

There is provided a novel pipeline for detection of a virus from clinical specimens. More particularly, the pipeline can be used to detect, for example, the SARS-CoV-2 virus.

### BACKGROUND OF THE INVENTION

Currently, two detection methods are widely used for pathogen screening and disease diagnosis, namely serological and molecular detection systems. SARS-CoV2 viral infections have lesser viral loads, especially in the early stages of the disease which is very difficult to detect with conventional detection methods. There is an imminent need to develop highly sensitive, accurate, fast, and low-cost detection systems. The lack of sensitivity of the current gold standards (RT-qPCR) in SARS-CoV2 infections requires special attention to reduce false negative rates in overall viral monitoring.

Another alternative approach for screening and detection of the SARS-CoV2 is based on loop-mediated isothermal amplification (LAMP) assay. Previous studies using LAMP methods have reported detection of few-copies which may be challenging for RT-qPCR based methods.

Centres for Disease Control and Prevention (CDC) suggests collection of specimens from the upper respiratory system, including a saliva sample (see for example, https://www.cdc.gov/coronavirus/2019-nCoV/lab/guidelines-clinical-specimens.html). Current laboratory procedures involve RNA isolation from the specimen, cDNA synthesis via reverse transcription, and amplification of the target region for detection.

### BRIEF SUMMARY OF THE INVENTION

Due to the high COVID-19 demand, the inventors were unable to obtain the required materials for viral detection. This inadequacy in supplies led to a finding of alternative ways of using LAMP reactions in which every single key component in the traditional system was reorganized to achieve operational LAMP protocols. The present invention skips the isolation step which decreases the overall cost and the test time by more than 20 minutes. This method can also be applicable for analysis of other viruses by designing new primer sets specific to the new target.

In one aspect of the present system, there is provided a LAMP-based detection kit that is highly sensitive making it the right detection method for high-throughput population screening and diagnosis efforts. The present invention is in the field of molecular detection systems, where the proposed process has a better resolution in screening the presence of a virus. The present invention provides a RT-LAMP based SARS-CoV2 detection pipeline that is optimized at every stage of the process to become a competent COVID-19 screening system.

In a further aspect of the present invention, there is provided a system which optimizes the virus detection procedure from saliva samples or other biological samples, and creates a highly sensitive, fast, accurate and low-cost detection kit. During the optimization, in one example embodiment the system uses;
a saliva/nasopharyngeal swab sample collection buffer,
a protocol which does not require an RNA-isolation step,
alternative approaches in detection of a virus using fluorescent or colorimetric screenings,
a design of primer mixture composed of six different oligonucleotides targeting COVID-19,
volume adjustment for providing an adequate template for polymerization.

While the detection kit can be used for detection of SARS-COV2, it can also be used for analysis of other viruses by designing new primer sets specific to the new target

In one aspect of the present invention there is provided a highly sensitive protocol that responds to low viral load, sensitive enough to be an alternative to the RT-qPCR method.

In a further aspect, there is provided a method for detection of a virus, comprising the steps of: collecting a sample from a patient; adding the sample into a buffer; heating the buffer containing the sample to cause lysis of the cells; enriching genetic material of the virus with super absorbent polymers; using reverse transcriptase to convert RNA to cDNA, and LAMP reaction with primer pairs; and evaluating results of the reaction; wherein the method does not use an RNA isolation step.

In yet a further aspect, there is provided a method for detection of a virus, comprising the steps of: providing a pre-isolated and purified RNA sample from a patient; using reverse transcriptase to convert the purified RNA to cDNA, and LAMP reaction with primer pairs; and evaluating results of the reaction.

### BRIEF DESCRIPTION OF THE DRAWINGS

The invention will be further understood from the following description with reference to the attached drawings.

FIG. 1 illustrates a result of a fluorometric RT-LAMP assay run for samples with duplicates of four different synthetic SARS-CoV2 RNA template concentrations ranging from 1.6 pg down to 1.6 fg which are 100,000 to 100 copies of viral RNA, respectively.

FIG. 2 illustrates a sample process for detecting the virus.

### DETAILED DESCRIPTION

A preferred embodiment of the present invention will be set forth in detail with reference to the drawings, in which like reference numerals refer to like elements or method steps throughout.

Current laboratory pipeline procedures for detection of a viral RNA through LAMP method involve collection and transport of the sample, lysis and release of the genetic materials, RNA isolation, cDNA synthesis via reverse transcription, and a LAMP reaction. The present invention skips the isolation step. For this purpose, there is provided a protocol wherein the viral RNA is concentrated and hence, not only removes the need of RNA purification but also increases the chance of catching viral particles for molecular testing.

In one example aspect of the present invention for collection of saliva samples, a decrease in the container size and solution volume using a 1.5 or 2 mL microcentrifuge tube as a container with Tris-EDTA solution in 50 ul volume were adopted. Additionally, use of a mild lysis buffer prevents inhibition of reverse transcriptase enzyme used to convert RNA to complementary DNA. For example, for 50 ul of 10×Tris-EDTA solution (200 mM Tris and 20 mM EDTA), 450 ul of saliva sample is collected in the microcentrifuge tube. Another example for collection of the nasopharyngeal swab samples, a swab is placed in a microcentrifuge tube containing 250 uL of 1×Tris-EDTA solution (20 mM Tris and 2 mM EDTA). By mixing manually or via a vortex, the sample is transferred from the swab to a lysis solution, and then the swab is discarded. For lysis of the cells and viruses from saliva and swab samples, a heat treatment at 95° C. for 5 minutes is applied. After cooling down to room temperature in 2-5 minutes, lysed samples are processed using a super absorbent polymer. In the present system, one example polymer that was used is MyMagiCon-RW100™ kit (Gigabomol Biotechnology, Turkey), other polymers with similar properties can also be used. This polymer absorbs most of the liquid. The flow-through, containing the concentrated viral RNA is transferred into another sterile DNase/RNase-free microcentrifuge tube without disturbing the swollen absorbent polymer. This step discards the possible inhibition of downstream methods resulted from cellular debris, and concentrates the genetic material of the virus. If the following procedures cannot be carried immediately, that concentrated sample can be stored at −20° C. or −80° C. If the end user has a pre-extracted and purified RNA sample, the steps above can be skipped. In other words, if the end user has pre-isolated and purified RNA, it can also be used by skipping the collection, heat treatment, and polymer steps.

In an example embodiment, the reverse transcription and LAMP reaction were carried simultaneously in the same reaction tube. For that purpose, an RT-LAMP mixture containing reverse transcriptase and Bst polymerase enzymes, such as WarmStart® LAMP Kit (New England Biolabs-Cat. No. E1700, USA), is used. It is also possible to synthesize cDNA from processed RNA with a suitable cDNA synthesis kit, and then this cDNA product can be used as a template in a separate LAMP reaction. For a typical RT-LAMP reaction carried using WarmStart® LAMP Kit as an example, 8 ul of WarmStart LAMP 2× Master Mix, 0.32 ul of fluorescent dye (50×), 0.64 ul of 25×LAMP primer reaction mixture (composed of 20 uM FIP, 20 uM BIP, 5 uM LoopF, 5 uM LoopB, 5 uM F3, and 5 uM B3 primers), 1 ul of processed saliva sample, and 6.04 ul of nuclease-free water are mixed, and reaction is carried on a suitable real-time fluorescent detection system at 65° C. for 140 cycles (15 sec/cycle) with proper excitation and emission parameters suitable for the fluorescent dye used in RT-LAMP reaction. The presented amounts can be optimized for any primers that are selected. The present system is also suitable to use other primers designed by the end user or other primer designs in the literature, such as the primer set targeting N region of SARS-CoV-2 RNA designed for a study (Butler et al., Nature Communications, 2021, https://doi.org/10.1038/s41467-021-21361-7) (Table 1). In this case, it may be necessary to optimize the final concentrations of the primers.

TABLE 1
One of the RT-LAMP primer mixtures found in the
literature to be used in RT-LAMP assay for SARS-
CoV-2 detection (Butler et al., 2021).

| Primer | Sequence |
|---|---|
| F3 | ACCAGGAACTAATCAGACAAG |
| B3 | GACTTGATCTTTGAAATTTGGATCT |
| FIP | TTCCGAAGAACGCTGAAGCGGAACTGATTACAAACATTGGCC |
| BIP | CGCATTGGCATGGAAGTCACAATTTGATGGCACCTGTGTA |
| LoopF | GGGGGCAAATTGTGCAATTTG |
| LoopB | CTTCGGGAACGTGGTTGACC |

In a further example embodiment for LAMP assay, two strategies are adopted for detection of the amplification; (i) evaluation of turbidity or change in colour of pH-sensitive dyes by the naked eye or (ii) detection of the signals coming from double-stranded DNA-binding fluorescent dye via a real-time detection device. Depending on the detection system, adding a fluorescent dye for fluorometric detection, or a pH-indicator for colorimetric detection, or without any additive for turbidity-based detection can be chosen. The reaction can be carried out at 65° C. for 30 minutes. The presence of a typical amplification curve for fluorescent detection, colour change in colorimetric detection, or formation of turbidity are examined for the evaluation step.

In one aspect of the present invention, thanks to the primers and sample processing procedure, there is provided high sensitivity and faster detection time within 7 to 40 minutes, depending on the viral load in the specimen. Colorimetric/turbidity-based detection alternative in this system is easy to use with no need for a fully equipped laboratory. By minor modifications, the procedure can be used for swab samples, or RNA material which has been isolated before. In the event of a mutation of the coronavirus, the system can be easily adapted through a change of primer sets. In addition to the SARS-CoV2 detection, the same process can be adapted to the detection of other viruses and pathogens from a wide range of industries including but not limited to health, food, and agriculture industries.

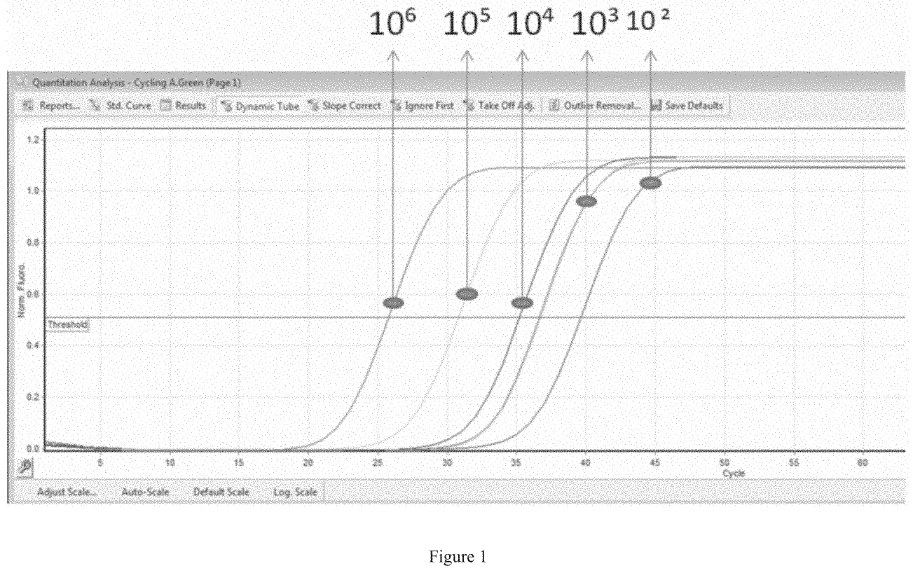

FIG. 1 shows an example of a result of a fluorometric RT-LAMP assay run for samples with five different synthetic SARS-CoV2 RNA template concentrations ranging from 1.6 pg (10⁶ copies of viral RNA) down to 1.6 fg (10² copies of viral RNA). As shown in FIG. 1, this system has a limit of detection as low as 1.6 fg of viral RNA in a reaction which roughly accounts for 100 copies. In this example, the high sensitivity requires careful sample treatment by the end-user. Although not necessary, it is recommended to carry out the protocol in BSL2 laboratories. The protocol in this example gives reproducible results whenever proper sampling is carried out.

In another aspect of the present invention, the protocol works best at a temperature of 65° C. with proper machinery that can keep the reaction temperature constant. For example, it is preferred for the reaction temperature to not fluctuate over +−0.1° C. as the continuous polymerization should be assured on the template.

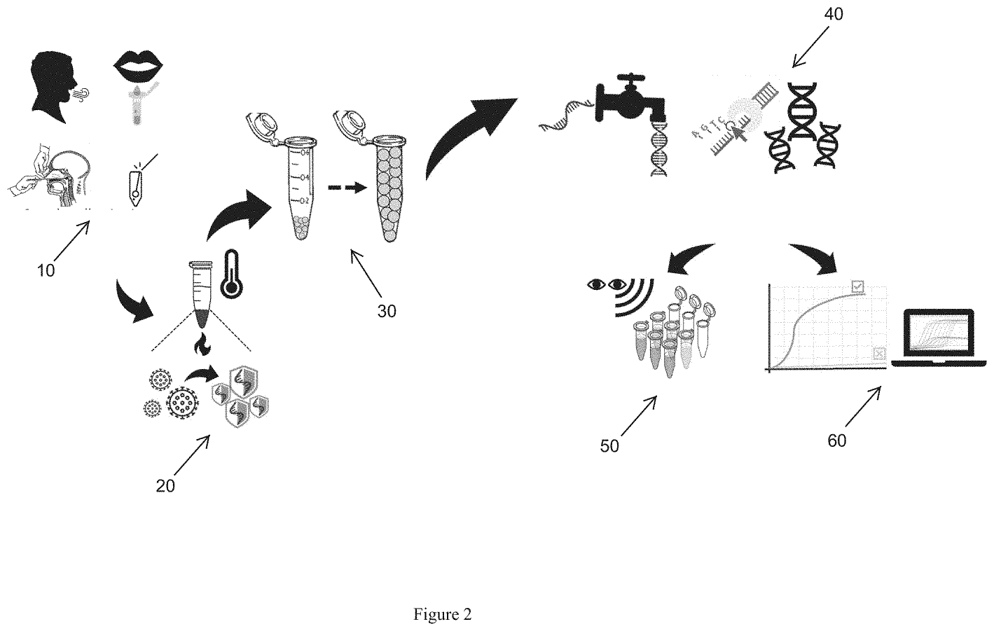

FIG. 2 shows a flow chart of the general steps in one example process of SARS-CoV2 detection via RT-LAMP assay in accordance with the present invention. A collection 10 of nasopharyngeal swab samples are taken and inserted into a tube container including a Tris-EDTA buffer. Lysis 20 of the cells within the specimen occurs with heat. Enrichment 30 of RNA molecules occurs with super absorbent polymers. cDNA synthesis 40 from RNA occurs by reverse transcriptase and application of target sequences by LAMP reaction. The result can be evaluated 50 by the naked eye based on colour change of the pH-indicator dye. As an alternative, or in addition, analysis 60 of the fluorescent signals via real-time analysis system can occur.

The scope of the claims should not be limited by the preferred embodiments set forth in the examples but should be given the broadest interpretation consistent with the description as a whole. For example, the possible sizes are also constructible. The amounts, sizes and example tests discussed herein are for example purposes only and should not limit the scope of the claims or variants thereof which would be understood by a person of skill in the art. Although it appears as a possible disadvantage, the operation cost will be logarithmic due to the required polymerization process. Furthermore, this method can also be applicable for analysis of other viruses by designing new primer sets specific to the new target.

What is claimed is:

1. A method for detection of a virus, comprising the steps of:
collecting a sample from a patient;
adding the sample into a buffer;
heating the buffer containing the sample to cause lysis of the cells;
enriching genetic material of the virus with super absorbent polymers;
using reverse transcriptase to convert RNA to cDNA, and LAMP reaction with primer pairs; and
evaluating results of the reaction;
wherein the method does not use an RNA isolation step.

2. The method of claim 1 wherein the step of sample collection includes saliva sample collection.

3. The method of claim 1 wherein the step of sample collection includes nasopharyngeal swab sample collection.

4. The method of claim 1 wherein the step of evaluating includes the step of using pH-indicator dye to determine colour change.

5. The method of claim 1 wherein the step of evaluating includes the step of analysing a fluorescent signal of the results using a real-time analysis system.

6. The method of claim 1 wherein the buffer is suitable for reverse transcription and LAMP reaction.

7. The method of claim 1 wherein the step of evaluating involves visually observing a colour change of a pH-indicator dye.

8. The method of claim 1 wherein the LAMP reaction uses three new primer pairs.

9. The method of claim 1 wherein the LAMP reaction is performed in a test tube with the reverse transcriptase.

10. The method of claim 8 wherein the LAMP reaction is performed in a test tube with the reverse transcriptase.

11. A method for detection of a virus, comprising the steps of:
providing a pre-isolated and purified RNA sample from a patient;
using reverse transcriptase to convert the purified RNA to cDNA, and LAMP reaction with primer pairs; and
evaluating results of the reaction.

12. The method of claim 11 wherein the RNA sample is obtained from a saliva sample.

13. The method of claim 11 wherein the RNA sample is obtained from a nasopharyngeal swab sample.

14. The method of claim 11 wherein the step of evaluating includes the step of using pH-indicator dye to determine colour change.

15. The method of claim 11 wherein the step of evaluating includes the step of analysing a fluorescent signal of the results using a real-time analysis system.

16. The method of claim 11 wherein the LAMP reaction uses three new primer pairs.

17. The method of claim 11 wherein the LAMP reaction is performed in a test tube with the reverse transcriptase.

18. The method of claim 16 wherein the LAMP reaction is performed in a test tube with the reverse transcriptase.


SEQUENCE LISTING (6 sequences)

SEQ ID NO: 1 - DNA, 21 bp, Artificial Sequence
  synthetic
accaggaactaatcagacaag

SEQ ID NO: 2 - DNA, 25 bp, Artificial Sequence
  synthetic
gacttgatctttgaaatttggatct

SEQ ID NO: 3 - DNA, 42 bp, Artificial Sequence
  synthetic
ttccgaagaacgctgaagcggaactgattacaaacattggcc

SEQ ID NO: 4 - DNA, 40 bp, Artificial Sequence
  synthetic
cgcattggcatggaagtcacaatttgatggcacctgtgta

SEQ ID NO: 5 - DNA, 21 bp, Artificial Sequence
  synthetic
gggggcaaattgtgcaatttg

SEQ ID NO: 6 - DNA, 20 bp, Artificial Sequence
  synthetic
cttcgggaacgtggttgacc